(19) United States
(12) Patent Application Publication
(10) Pub. No.: US 2004/0242619 A1
(43) Pub. Date: Dec. 2, 2004

(54) Processes for preparation of polymorphic forms of desloratadine

(76) Inventors: Zoltan G. Toth, Debrecen (HU); Viktor Gyollai, Debrecen (HU); Adrienne Kovacsne-Mezei, Debrecen (HU); Csaba Szabo, Debrecen (HU); Judith Aronhime, Rehovot (IL); Claude Singer, Kfar Saba (IL)

Correspondence Address: KENYON & KENYON, ONE BROADWAY, NEW YORK, NY, 10004, US

(21) Appl. No.: 10/800,290

(22) Filed: Mar. 12, 2004

Related U.S. Application Data
(60) Provisional application No. 60/526,339, filed on Dec. 1, 2003.
(60) Provisional application No. 60/516,904, filed on Nov. 3, 2003.
(60) Provisional application No. 60/515,354, filed on Oct. 28, 2003.
(60) Provisional application No. 60/454,299, filed on Mar. 12, 2003.

(51) Int. Cl. C07D 221/22; A61K 31/473

(52) U.S. Cl. 514/290; 546/93

(57) ABSTRACT

Provided are processes for preparation of polymorphic forms of desloratadine.

### PRIORITY

This application claims the benefit of U.S. provisional application Ser. No. 60/526,339, filed Dec. 1, 2003, U.S. provisional application Ser. No. 60/516,904, filed Nov. 3, 2003, U.S. provisional application Ser. No. 60/515,354, filed Oct. 28, 2003, and U.S. provisional application Ser. No. 60/454,299, filed Mar. 12, 2003, the contents of all of which are incorporated herein.

### FIELD OF THE INVENTION

The present invention relates to the solid state chemistry of desloratadine.

### BACKGROUND OF THE INVENTION

Desloratadine, known as 8-chloro-6,11-dihydro-11-(4-piperidylidene)-5H-benzo[5,6]cyclohepta[1,2-b]pyridine, has the following structure:

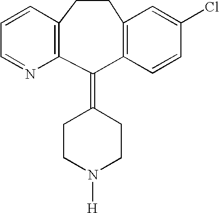

and is disclosed in U.S. Pat. No. 4,659,716. Desloratadine is currently marketed as Clarinex® in the United States. Clarinex is prescribed as an antihistamine for prevention or treatment of allergenic reactions, which may result in symptoms such as sneezing, itchy eyes and hives. The '716 patent discloses methods for preparing and administering desloratadine and its pharmaceutically acceptable salts, and is incorporated herein by reference. See also U.S. Pat. No. 4,282,233, incorporated herein by reference, which discloses loratadine.

The present invention relates to the solid state physical properties of desloratadine. These properties can be influenced by controlling the conditions under which desloratadine is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate. Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

In Example V, the '716 patent prepares desloratadine in the solid state and discloses: “Extract the organic material with chloroform, wash with water and remove the solvent. Triturate the residue with hexane. Recrystallize from a large volume of hexane after charcoal decolorization to obtain the product, m.p. 151′-152° C.”

In Example VI, B, desloratadine is also prepared in the solid state: “The material is extracted several times with chloroform, the chloroform extracts washed with water and concentrated to dryness, and the residue triturated with petroleum ether or hexane to yield 11.5 grams (93%) m.p. 149°-151° C. After recrystallization from hexane, the product melts at 150°-151° C.” The starting material for Example VI, B, is an N-cyano compound prepared according to the disclosure in U.S. Pat. No. 3,326,924.

Both U.S. Pat. No. 4,282,233 and U.S. Pat. No. 3,326,924 are incorporated herein by reference, particularly for their disclosure of preparation of desloratadine.

U.S. Pat. No. 6,506,767 discloses two polymorphic forms of desloratadine, labelled Forms I and II (syn. form 1 and form 2). The XRPD peaks and the FTIR spectrum for the forms are also disclosed in the '767 patent.

The '767 patent discloses: “Surprisingly we discovered that certain alcoholic solvents, e.g., hexanol and methanol produced 100% polymorph form 1, but others, e.g., 3-methyl-1-butanol and cyclohexanol produced significant amounts of form 2. Chlorinated solvents, e.g., dichloromethane produced form 1 substantially free of form 2 but the compounds were discolored. Ether solvents such as dioxane produced form 1 substantially free of form 2 but other alkane ethers,, e.g., di-isopropyl ether produced form 1 with significant amounts of form 2 and di-n-butyl ether favored formation of form 2. Ketones such as methyl isobutyl ketone produced crystalline polymorph form 1 essentially free of Form 2 but methyl butyl ketone produced a 8:1 ratio of form 1 to form 2. Use of methyl isubutyl ketone is preferred to produce crystalline polymorph form 1 essentially free of form 2. Only ethyl acetate and di-n-butyl ether were found to produce crystalline polymorph form 2 substantially free of form 1. Use of di-n-butyl ether is preferred for producing crystalline form 2 substantially free of form I.”

The '767 patent, in Examples 1-3, prepares Form I by crystallization out of methyl isobutyl ketone, while in examples 4 and 5, prepares Form II by crystallization out of ethyl acetate and di-n-butyl ether, respectively.

The '767 patent also carried out stability tests on Polymorph Form I. According to the '767 patent, Form I was “subjected to stability testing at various temperatures (25, 30 and 40° C.) and relative humidities of 60%, 60% and 75%, respectively . . . No significant change (<1%) from initial sample % form 1 and related compounds was observed.”

The '767 patent warns against using polymorphic mixtures of desloraratadine for formulation. According to the '767 patent, “such a mixture could lead to production of a [desloratadine] which would exist as a variable mixture of variable composition (i.e., variable percent amounts of polymorphs) having variable physical properties, a situation unacceptable in view of stringent GMP requirements.”

The '767 patent is incorporated herein by reference in its entirety, and more particularly in respect to its characterization of the polymorphic forms, synthesis of the starting material and preparation of the various polymorphic forms.

There is a need in the art for additional processes for preparation of polymorphic forms of desloratadine.

### SUMMARY OF THE INVENTION

In one aspect, the present invention provides a process for preparing crystalline desloratadine Form I comprising the steps of preparing a solution of desloratadine in a solvent selected from the group consisting of acetonitrile, di-methyl form amide, tetrahydrofuran and diethylcarbonate, wherein desloratadine Form I crystallizes out of the solution and recovering the desloratadine Form I.

In another aspect, the present invention provides a process for preparing crystalline desloratadine Form I comprising the steps of preparing a solution of desloratadine in a solvent selected from the group consisting of chloroform and ethyl acetate, combining the solution with an anti-solvent to precipitate desloratadine Form I and recovering desloratadine Form I.

In another aspect, the present invention provides a process for preparing crystalline desloratadine Form I comprising the step of preparing a solution of desloratadine in a C₁ to C₄ alcohol, combining the solution with water to precipitate desloratadine Form I and recovering desloratadine Form I.

In another aspect, the present invention provides a process for preparing crystalline desloratadine Form I comprising the steps of preparing a solution of desloratadine in isopropanol, wherein desloratadine Form I precipitates from the solution; and recovering the desloratadine Form I.

In another aspect, the present invention provides a process for preparing crystalline desloratadine Form II comprising the steps of melting desloratadine to obtain a molten material, cooling the molten material to obtain a solid and grinding the solid.

In another aspect, the present invention provides a process for preparing a mixture of crystalline desloratadine Form I and Form II comprising the step of grinding crystalline desloratadine Form I.

In another aspect, the present invention provides a process for preparing crystalline desloratadine Form II comprising the steps of preparing a solution of desloratadine in dimethyl carbonate, wherein desloratadine Form II precipitates from the solution and recovering the desloratadine.

In another aspect, the present invention provides a process for preparing crystalline desloratadine Form I comprising the steps of preparing a solution of desloratadine in i-butyl acetate, wherein Form I precipitates from the solution and recovering the precipitate

In another aspect, the present invention provides a process for preparing crystalline desloratadine Form I comprising the steps of preparing a solution of desloratadine in a solvent selected from the group consisting of isopropanol and i-butanol, wherein desloratadine Form I precipitates from the solution and recovering the mixture.

In another aspect, the present invention provides a process for preparing a mixture of crystalline Form I and Form II of desloratadine comprising the step of drying desloratadine Form I crystals obtained by crystallization from a C1 to a C₄ alcohol.

In another aspect, the present invention provides a process for making a mixture of crystalline desloratadine Form I and Form II comprising the steps of combining a solution of desloratadine in a suitable solvent with an anti-solvent containing seeds of both Form I and Form II of desloratadine to precipitate the mixture, and recovering the mixture.

In another aspect, the present invention provides a process for preparing a mixture of desloratadine crystalline Forms I and II containing at least about 25% of both of the Forms comprising the steps of preparing a solution of desloratadine in a solvent selected from the group consisting of ethyl acetate and iso-butyl acetate, in a mixture with about 3% to about 20% C₁ to C₄ alcohol by volume, wherein the mixture of Form I and II precipitates from the solution and recovering the mixture.

In another aspect, the present invention provides a process for preparing a mixture of crystalline desloratadine Form I and II comprising the steps of preparing a solution of desloratadine in iso-butyl acetate, combining the solution with a C₅ to C₁₂ aromatic hydrocarbon to precipitate the mixture, wherein the combining may be carried out before, after or during crystallization and recovering the mixture.

In another aspect, the present invention provides a process for preparing a mixture of crystalline desloratadine Form I and II comprising the steps of preparing a solution of desloratadine in iso-butyl acetate, combining the solution with iso-butyl acetate at a temperature lower than the solution to crystallize the mixture and recovering the mixture.

In another aspect, the present invention provides a process for preparing a mixture of crystalline desloratadine Form I and Form II comprising the steps of preparing a solution of desloratadine in ethyl acetate, seeding the solution with a mixture of Form I and Form II, combining the solution with a C₅ to C₁₂ saturated hydrocarbon, wherein the combining may be carried out before, after or during crystallization and recovering the mixture of desloratadine Form I and II.

In another aspect, the present invention provides a process for preparing a mixture of crystalline desloratadine Form I and Form II comprising the steps of preparing a solution of desloratadine in 2-propanol and toluene, wherein the mixture of Forms I and II precipitates from the solution and recovering the mixture.

In another aspect, the present invention provides a process for preparing a mixture of Form I and Form II, comprising the steps of providing a first solution of desloratadine in toluene, evaporating the toluene to obtain a residue, dissolving the residue in a mixture of toluene and a C₁ to C₄ alcohol to obtain a second solution, cooling the second solution to obtain a slurry, combining the slurry with a C₅ to C₁₂ saturated hydrocarbon to precipitate the mixture and recovering the mixture.

In another aspect, the present invention provides a process for preparing a mixture of desloratadine Form I and Form II comprising the steps of combining desloratadine acetate, toluene and KOH to obtain a reaction mixture, heating the mixture, whereby two phases are obtained, separating the phases, concentrating the separated organic phase, dissolving the obtained concentrate in a toluene-2-propanol mixture containing less than about 20% 2-propanol by volume, cooling the solution to obtain a slurry, combining the slurry with cold n-heptane and recovering mixture of desloratadine forms I and II.

In another aspect, the present invention provides a process for preparing crystalline desloratadine Form II comprising the steps of crystallizing desloratadine from toluene, and recovering the crystalline form.

### BRIEF DESCRIPTION OF THE FIGURE

FIG. 1 is a stability study of a polymorphic mixture of desloratadine.

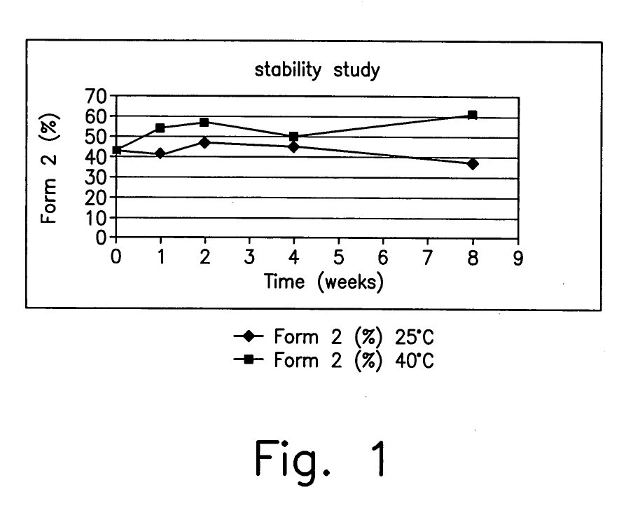

FIG. 2 is a DSC thermogram of desloratadine Form II after grinding and sieving.

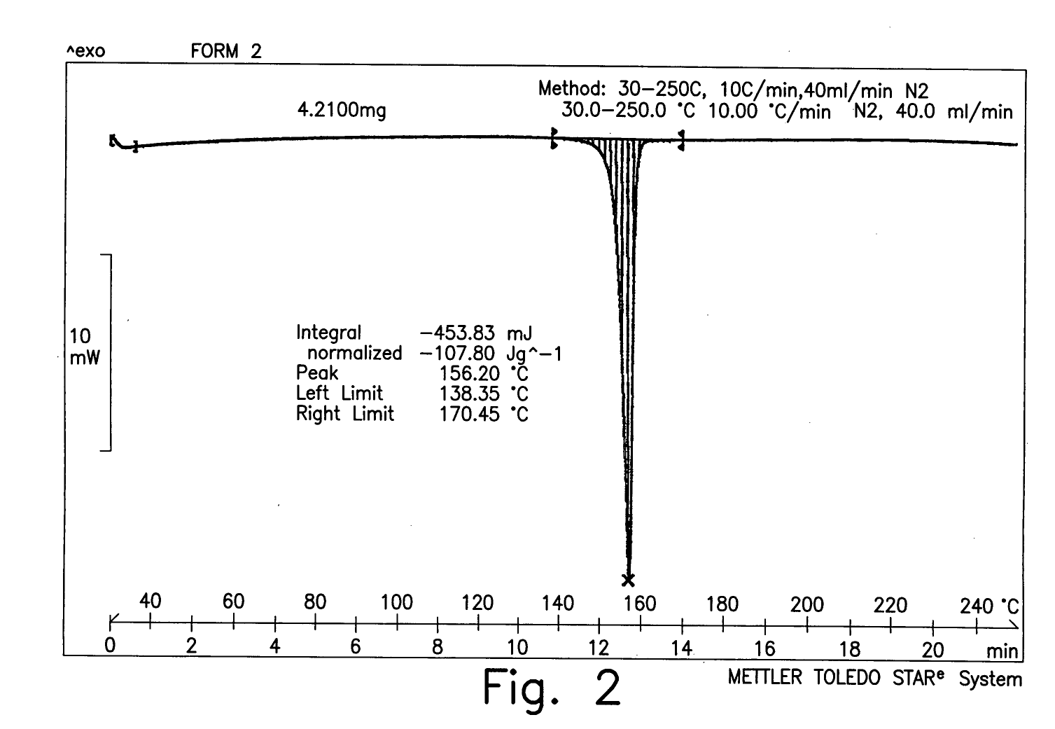

FIG. 3 is a DSC thermogram of desloratadine Form I after grinding and sieving.

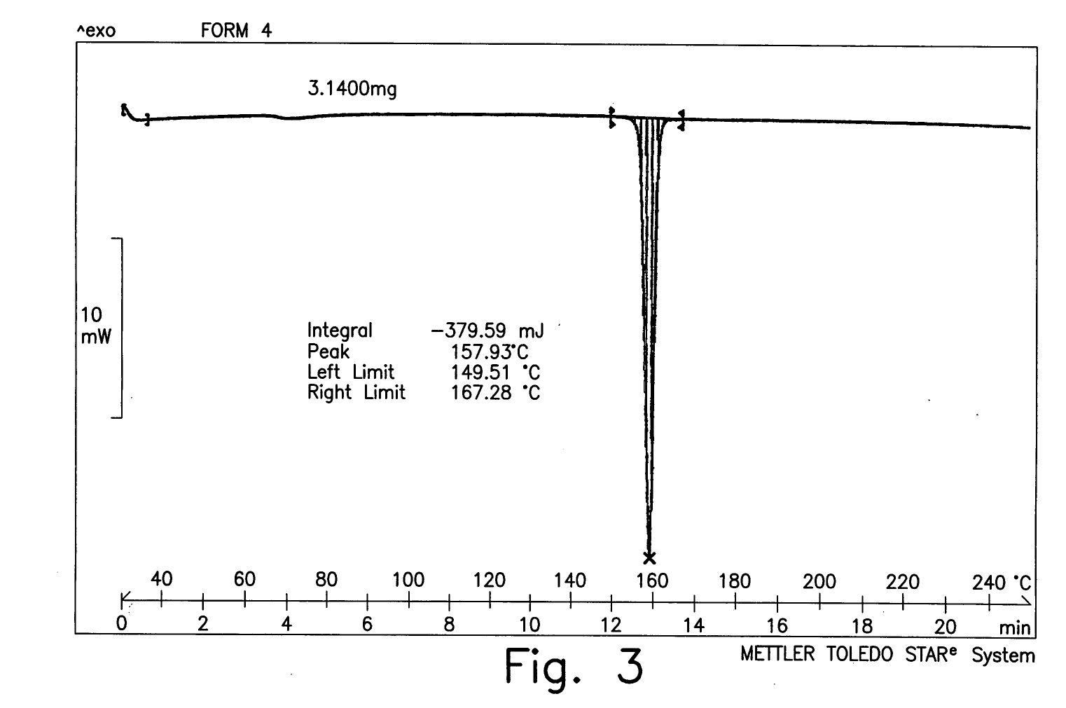

FIG. 4 is a DSC thermogram of a 25:75 mixture of Form I and Form II by weight.

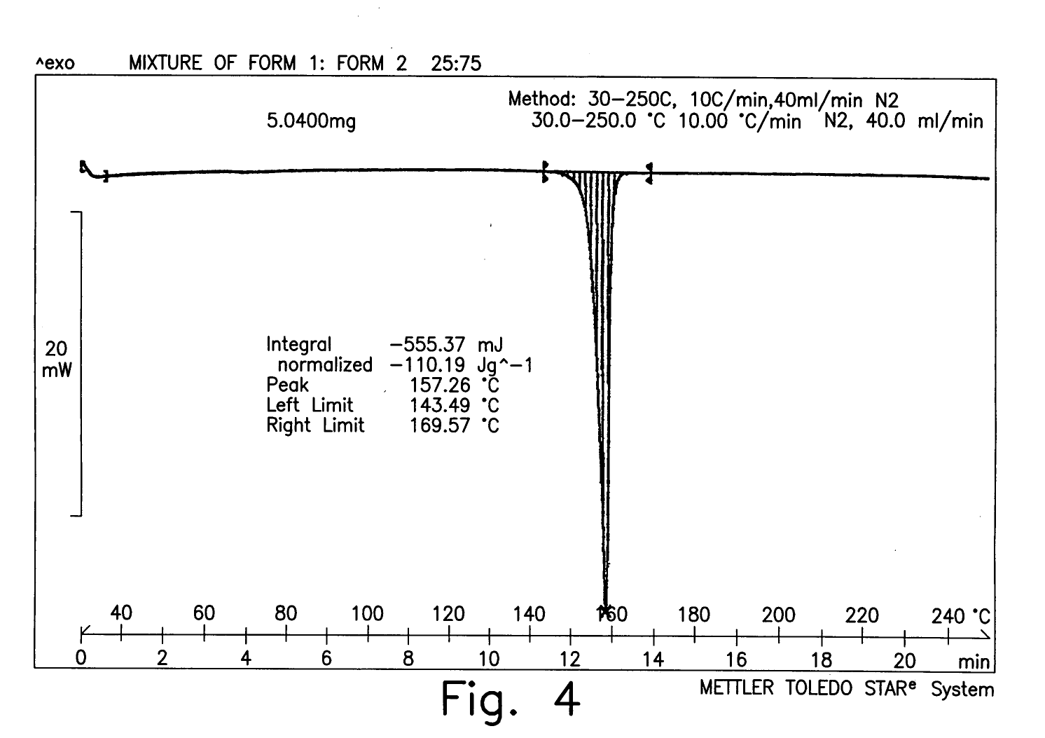

FIG. 5 is a DSC thermogram of a 50:50 mixture of Form I and Form II by weight.

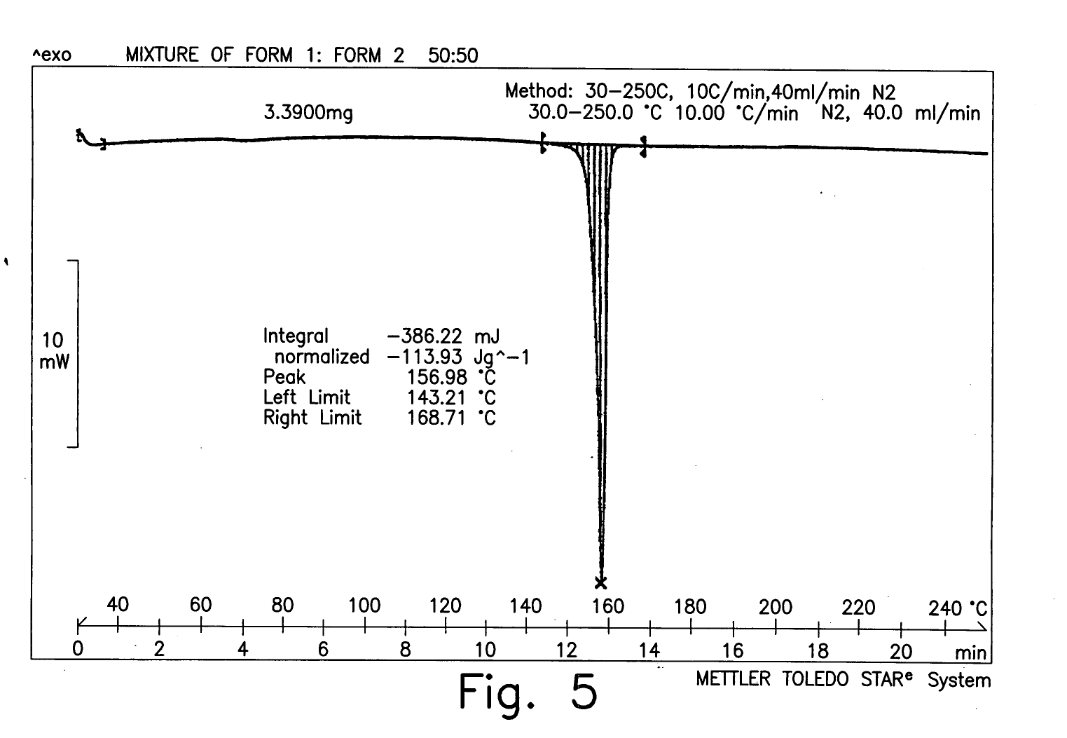

FIG. 6 is a DSC thermogram of a 75:25 mixture of Form I and Form II by weight.

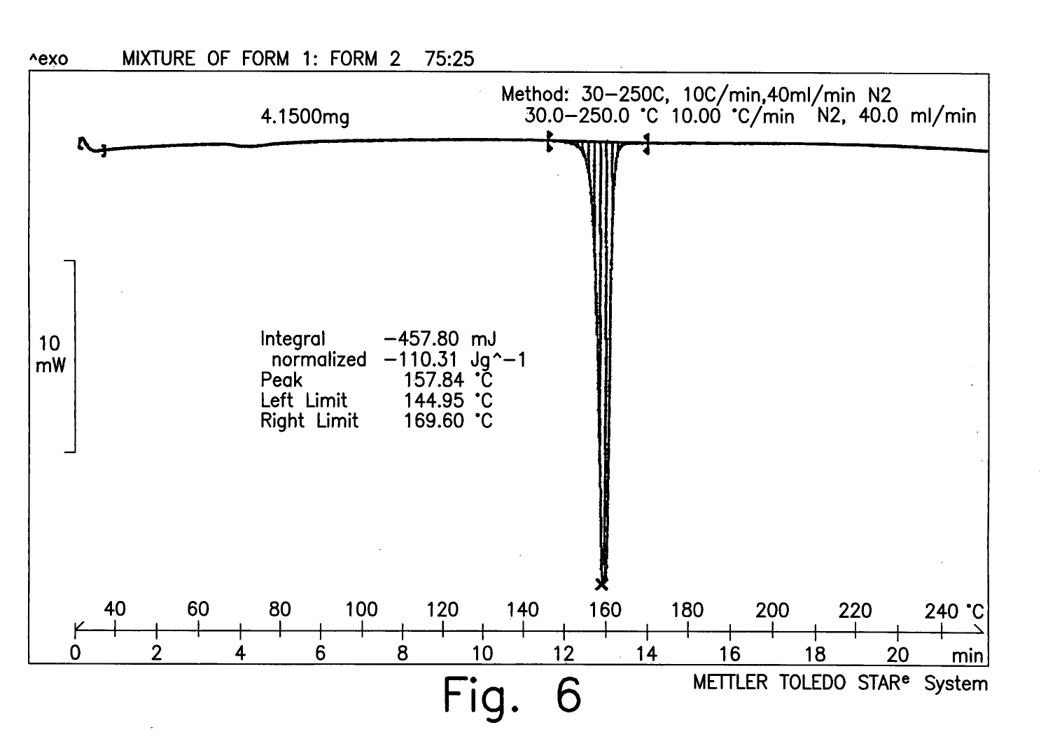

FIG. 7 is a DSC thermogram of a 84:16 mixture of Form I and Form II by weight.

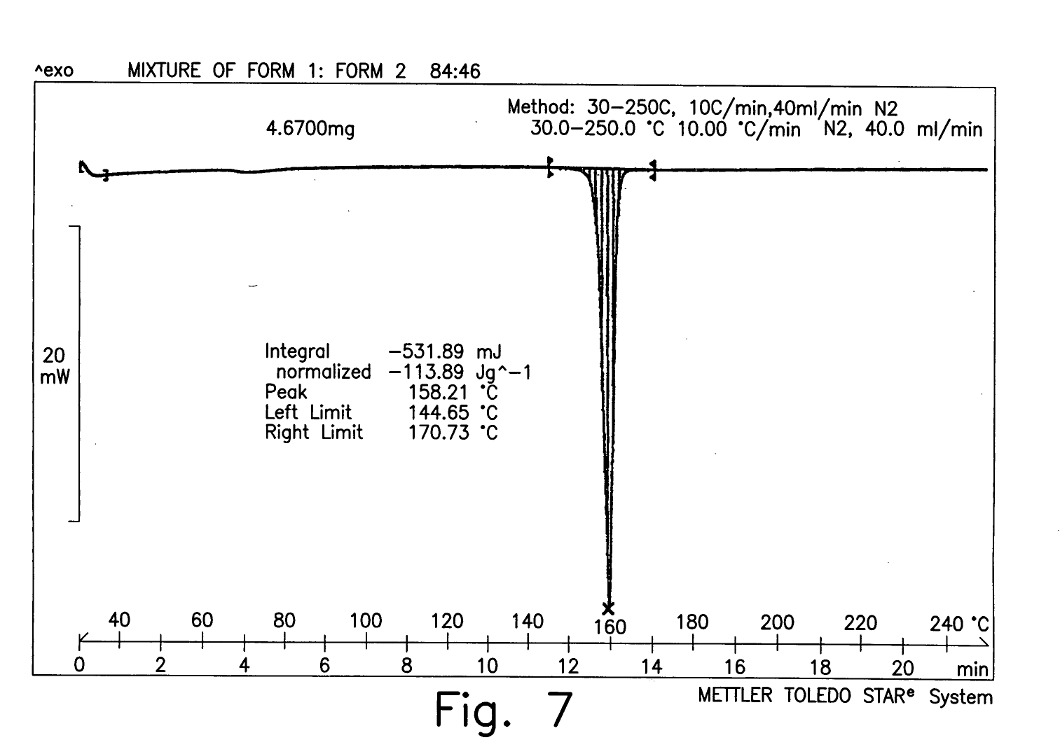

FIG. 8 is a comparison of X-ray powder diffraction patterns of desloratadine Form I and Form II, and various mixtures thereof.

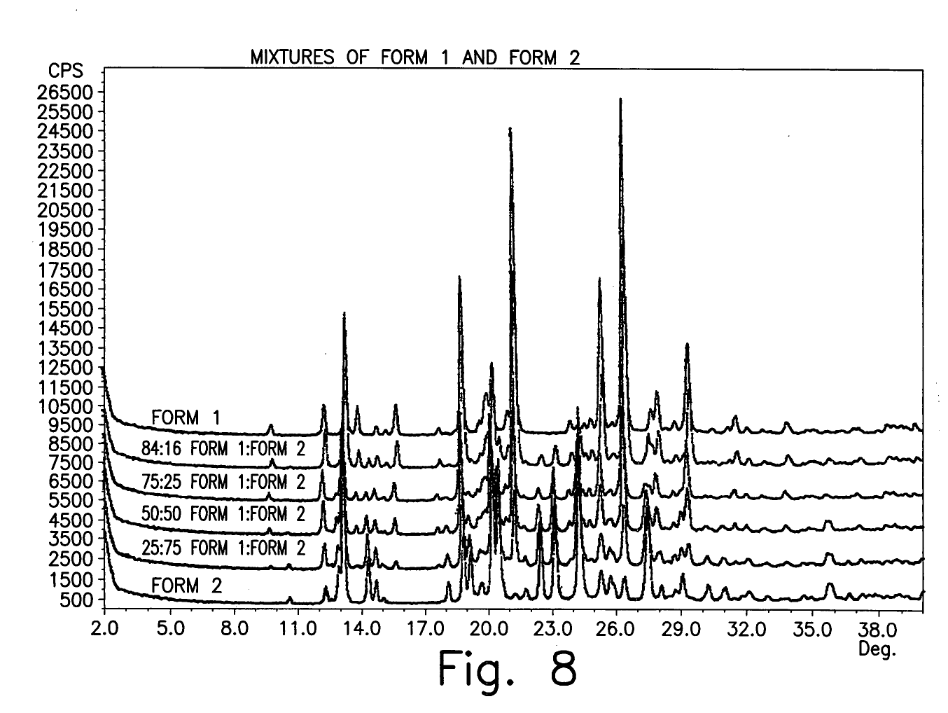

FIG. 9 is similar to FIG. 8, but illustrates the X-ray diffraction patterns after grinding.

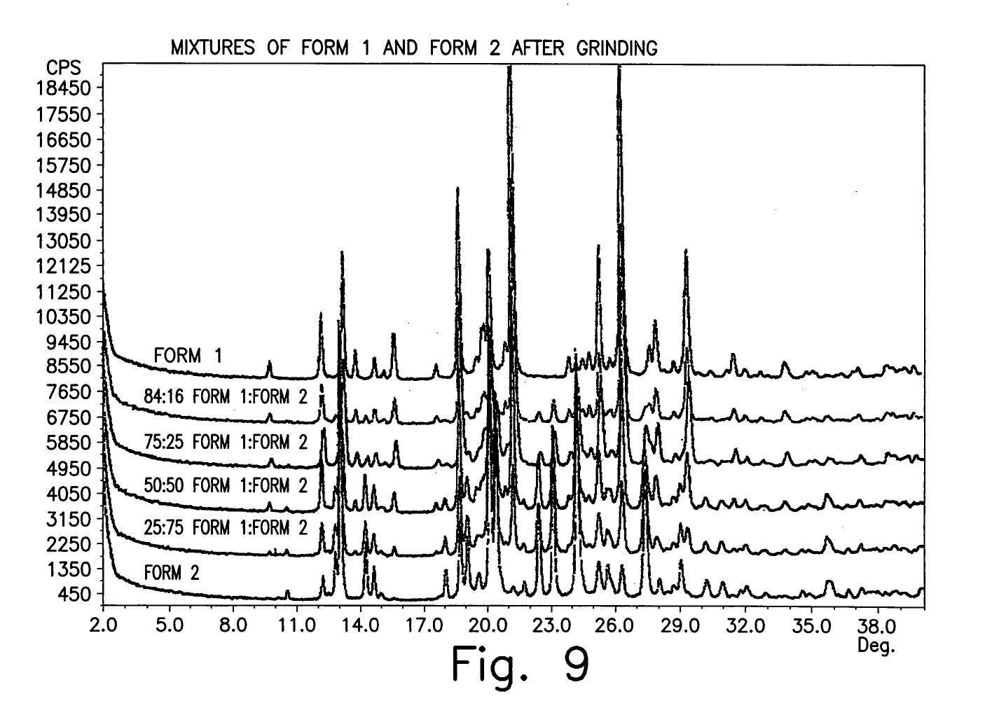

FIG. 10 is similar to FIG. 8, but illustrates the X-ray diffraction patterns after storage at 100% relative humidity.

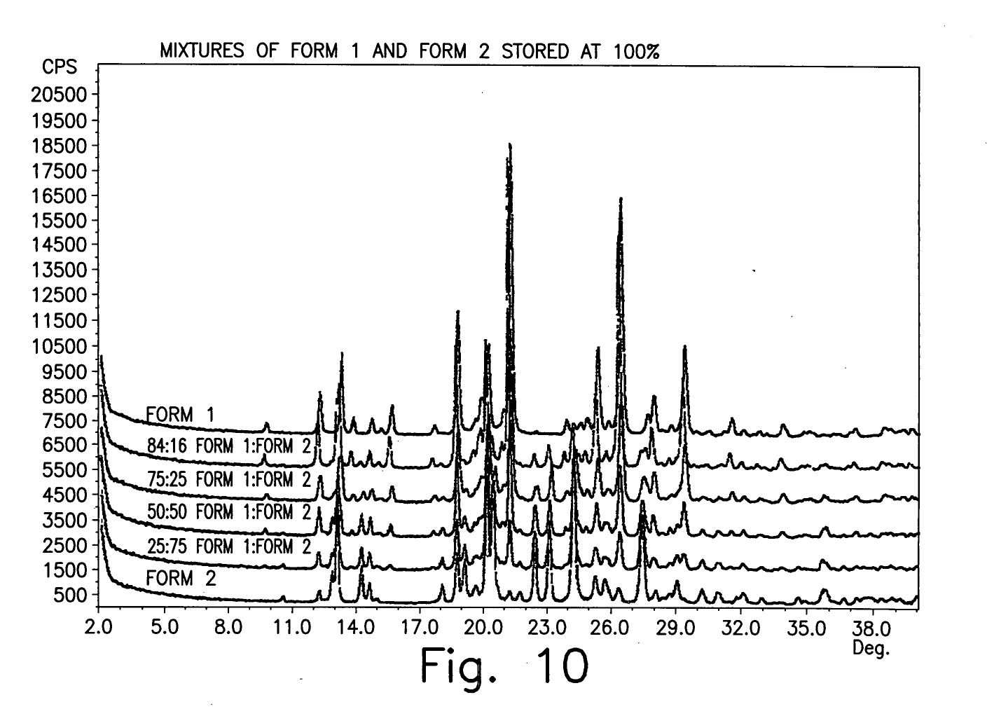

FIG. 11 is similar to FIG. 8, but illustrates the X-ray diffraction patterns after storage at 80% relative humidity.

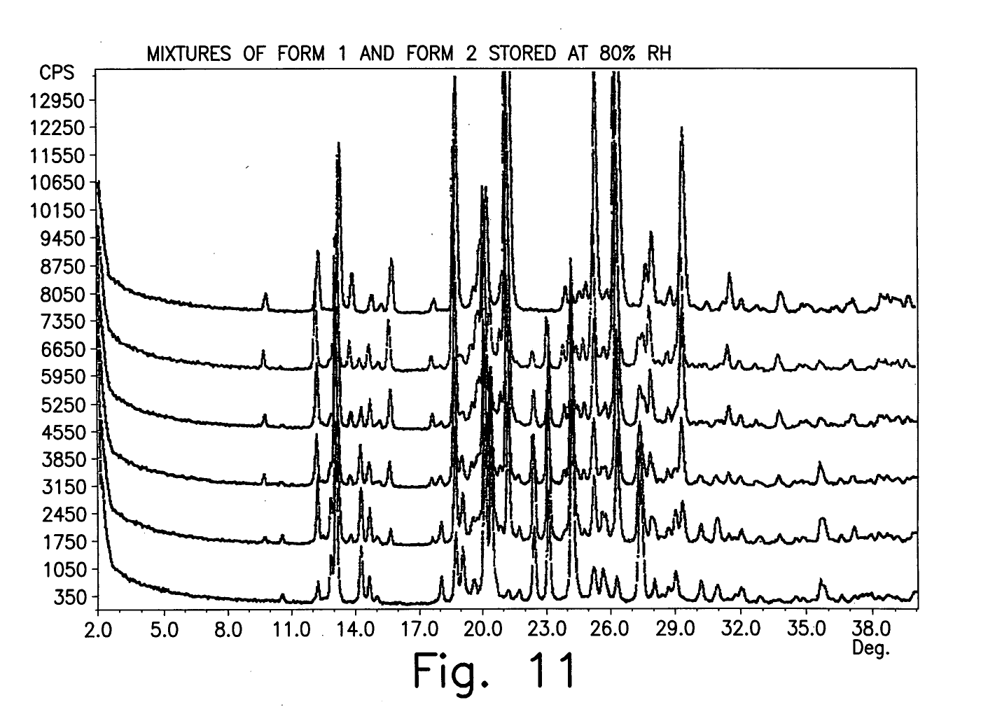

FIG. 12 is similar to FIG. 8, but illustrates the X-ray diffraction patterns after storage at 60% relative humidity.

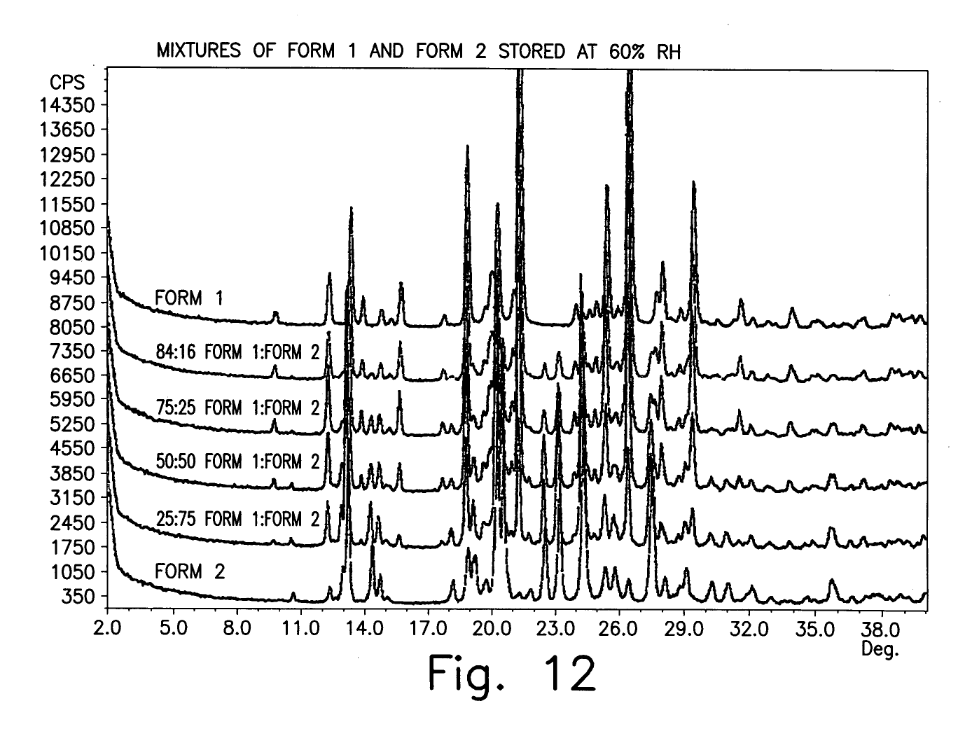

### DETAILED DESCRIPTION OF THE INVENTION

As used herein, the term drying refers to removal of solvent from a solid through application of heat.

As used herein, the term C₅ to C₁₂ saturated hydrocarbon refers to a straight/branched and/or cyclic/acyclic hydrocarbon. Preferred hydrocarbons are cyclohexane, cycloheptane, cyclohexane, n-heptane and n-hexane, with n-hexane and n-heptane being preferred. The terms hexane and heptane used hereinafter refer to n-hexane and n-heptane.

As used herein, the term C₅ to C₁₂ aromatic refers to substituted and unsubstituted hydrocarbons having a phenyl group as their backbone. Preferred hydrocarbons include benzene, xylene and toluene, with toluene being more preferred.

As used herein, the term C₃ to C₇ ester refers to an ester having such number of carbons. Preferred esters include ethyl acetate.

As used herein, an anti-solvent is a liquid that when added to a solution of X in the solvent, induces precipitation of X. Precipitation of X is induced by the anti-solvent when addition of the anti-solvent causes X to precipitate from the solution more rapidly or to a greater extent than X precipitates from a solution containing an equal concentration of X in the same solvent when the solution is maintained under the same conditions for the same period of time but without adding the anti-solvent. Precipitation can be perceived visually as a clouding of the solution or formation of distinct particles of X suspended in the solution or collected at the bottom the vessel containing the solution.

The amount of Form I and Form II is expressed herein as a weight ratio relative to each other, i.e., (Form I or II)/Form I plus Form II×100%.

The present invention provides various processes for preparing polymorphic Forms I and II of desloratadine, and mixtures thereof.

Desloratadine may be crystallized as a mixture of polymorphs in such a way that the ratio between the polymorphs is consistent. As used herein, a “consistent ratio” (or consistent mixture) refers to a ratio of Form I compared to Form II (wt/wt) that is between a range of about ±10% (wt/wt) between lots, as measured by XRPD or FTIR.

In one embodiment, a mixture of desloratadine Form I and Form II is precipitated out of a suitable solvent such as chloroform or ethyl acetate by addition of an anti-solvent. Desloratadine is dissolved to an organic solvent such as chloroform or ethyl acetate.

Dissolution may be carried out by adding desloratadine to the solvent and heating the solvent to obtain a clear solution. A suitable anti-solvent is then added to precipitate the mixture. Examples of such anti-solvents include C₅ to C₁₂ saturated hydrocarbons, preferably saturated aliphatic hydrocarbons such as hexane and heptane, with hexane being more preferred. Another example of an anti-solvent is an ether whose alkyl radical groups connected to the oxygen atom are independently selected from the group consisting of methyl, ethyl, propyl, isopropyl, butyl, 1-butyl, 2-butyl and t-butyl. Most preferably, the ether is di-isopropyl ether. Preferred ethers are MTBE, di-isopropyl ether and methyl ethyl ether, with diisopropyl ether being more preferred.

The anti-solvent is then combined with the solution, preferably by being added to the solution, to precipitate desloratadine. Preferred combinations of solvent/anti-solvent include chloroform/hexane or diisopropyl ether, or alternatively ethyl acetate/hexane. The ratio in the final product of the reaction might vary depending on the solvent/anti-solvent used, crystallization temperature and the temperature of the solution. When hexane is added to a solution of chloroform having a temperature above about 40° C., followed by crystallization at a temperature of from about 20° C. to about 30° C., the product contains from about 35% to about 40% Form II, in comparison to Form I. Preferably, the temperature of the solution is above about 40° C. for chloroform, more preferably from about 40° C. to about reflux (69° C.), and most preferably about 45° C. to about 55° C. When hexane or diisopropyl ether is added to chloroform having a temperature of below about 40° C., more preferably from about 20° C. to about 30° C., followed by cooling to a temperature of from about 0° C. to about 10° C., the product contains from about 2% to about 6% Form II. If ethyl acetate is used as a solvent, followed by addition of cold hexane and crystallization at a temperature below about 0° C., the resulting product has From II in the range of from about 15% to about 25%.

The resulting precipitate may then be recovered by techniques well known in the art, such as filtration, and optionally dried.

Form I having about a 2% to about 10%, more preferably about 4% Form II, may also be obtained by precipitation out of a C₁ to C₄ preferably ethanol through use of water as an anti-solvent. Water is preferably combined with ethanol at a temperature of from about 20° C. to about 30° C., followed by cooling to a temperature of from about 0° C. to about 10° C. The resulting precipitate may be recovered by techniques known in the art, and may optionally be dried.

Form I in a mixture with about 15% to about 25% of Form II may also be prepared by crystallization out of i-butyl acetate. A solution of desloratadine is prepared in i-butyl acetate, followed by crystallization. Preferably the solution is heated to reflux, followed by cooling to a temperature of from about 20° C. to about 30° C. The resulting crystals may then be recovered by techniques well known in the art, such as filtration, centifugation, decanting. The recovered crystals may also be dried.

A mixture of the two forms or Form I with less than about 10% Form II may also be prepared by crystallization out of isopropanol or i-butanol. A solution of desloratadine is prepared in isopropanol or i-butanol, followed by crystallization. Preferably, a solution is obtained by heating, followed by cooling to a temperature of about −101C to about 30° C., more preferably from about 0° C. to about 25° C. When isopropanol is a solvent, cooling to a temperature of from 20° C. to about 30° C. results in about 5-6% Form II, while cooling to a temperature of from about −10C to about 10° C. results in about 2% of Form II. However, seeding with Form II increases the ratio of Form II to Form I. With isobutanol, cooling to a temperature of from about 20° C. to about 30° C. results in about 3% Form II. Thus, the present invention provides a process for increasing the amount of Form I by decreasing crystallization temperature. The resulting crystals may then be recovered by techniques well known in the art, such as filtration, centrifugation and decanting. The recovered crystals may also be dried. The drying results in a mixture that is approximately a 50:50 mixture.

When crystals obtained from crystallization from isopropanol and isobutanol are dried by storage at room temperature, a transition to a 1:1 mixture of Form I to Form II occurrs, despite the starting product having a much higher ratio of Form I to Form II. This transition suggests that a 50:50 mixture may be prepared by storing crystals obtained from at least C₁ to C₄ alcohols. Preferably, storage is carried out at a temperature of from about 20° C. to about 30° C., under reduced pressure. As used herein, the term reduced pressure refers to a pressure below about 100 mmHg, more preferably of about 10 mmHg to about 50 mmHg.

The present invention also provides a process for preparing a mixture of desloratadine forms I and II by grinding desloratadine Form I. The duration of the grinding may vary depending on the desired final product and how the grinding is carried out. One of skill in the art may take an XRD after grinding to determine the optimal amount of time for grinding. Preferably, the grinding is carried out from about ½ an hour to about 3 hours. Usually, about 1 hour is sufficient to cause a transformation of Form I to Form II, i.e., more than about a 30% transformation of Form I by weight into Form II. In Example 14, the grinding results in about a 60% to about 70% mixture of Form II compared to Form I, more specifically about a 2:1 mixture. Substantially no polymorphic transformation occurred when grinding a mixture for up to about one minute. The grinding may be done by methods known in the art, such as manually in a mortar with pestle, or with any pressure generating device, such as a press.

Desloratadine may also be prepared in Form I or Form II, substantially free of the other form. As used herein, substantially free refers to having at most traces of the other form, i.e., less than about 1% weight of one polymorph to the other, more preferably less than about 0.5%, and most preferably less than about 0.1%. For example, Form I substantially free of Form II may be prepared by crystallization out of a suitable solvent. Desloratadine may be dissolved in an organic solvent such as acetonitrile, dimethylformamide, tetrahydrofuran and diethylcarbonate. The solvent may be heated to obtain a clear solution, preferably to reflux temperature (DMF is generally heated below its reflux temperature due to its high boiling point). Form I may then be recovered after crystallization. Crystallization may be induced for example by cooling the solvent, preferably to a temperature of from about 20° C. to about 30° C. The resulting crystals may then be dried under reduced or ambient pressure, preferably with slight heating.

Desloratadine Form II may also be prepared substantially free of Form I. In one embodiment, desloratadine is melted at a temperature above its melting temperature. The resulting molten material may then be allowed to cool naturally, such as at room temperature, or with a cooling apparatus to accelerate the cooling process. After cooling, preferably to a temperature of from about 20° C. to about 30° C., the molten material crystallizes into Form II substantially free of Form I. The resulting solidified material is preferably grounded.

Form II substantially free of Form I may be prepared by crystallization out of dimethylcarbonate. A solution of desloratadine in dimethylcarbonate is prepared, preferably by heating the dimethylcarbonate to about reflux temperature. The solution is then preferably cooled to a temperature of from about 20° C. to about 30° C. The resulting crystals may then be separated by techniques well known in the art, and optionally dried.

In another embodiment, a mixture of the two forms, i.e, from about a 35 to about 65% of both of the, may be prepared through combining a solution with a seeded antisolvent. In this embodiment, an anti-solvent is seeded with a mixture of desloratadine Form I and II, preferably a substantially equal mixture of the forms, and combined with a solution of desloratadine in a suitable solvent. A suitable solvent is isobutyl acetate. A suitable anti-solvent is a C₅ to C₁₂ aromatic or saturated hydrocarbon such as toluene or heptane.

The seeding allows for manipulating the ratio of Form I and II relative to each other. In example 19, the 1-butyl acetate/heptane procedure without seeding produced 63% Form I, while the same procedure with seeding produced 37% and 42% Form I in examples 17 and 18 respectively. The seeding also allows for crystallization of a consistent ratio in that examples examples 17 and 18 are well within the 10% margin.

A mixture of two polymorphic forms, i.e., from about 25 to about 75% of form of both forms, may be obtained by crystallization from a mixture of a solvent of desloratadine (e.g. toluene, ethyl acetate, iso-butyl acetate) and a co-solvent (a C₁ to C₄ alcohol such as iso-propanol and methanol). The polymorphic form ratio of the final product may be influenced by amount of the co-solvent. Preferably, the amount of the co-solvent is from about 3% to about 13% by volume, more preferably from about 7% to about 10% by volume. In example 2122, 23 and 24 different solvents are used to obtain a mixture of polymorphic forms. In example 21, 10% iso-propanol-iso-butyl acetate is used. In example 22, 10% iso-propanol-toluene is used. In example 23, 5% methanol-toluene is used. In example 24, 3% iso-propanol is used.

The use of a co-solvent, particularly a C₁ to C₄ alcohol, allows for manipulating ratio of Form II compared to Form I. Crystallization out of i-butyl acetate for example results in about 15-25% of Form II, while with addition of 10% isopropanol results in about 40-50% Form II. Crystallization out of ethyl acetate results almost entirely in Form II, while use of 3% isopropanol as a co-solvent allows for obtaining about 70-80% of Form I.

One skilled in the art may also appreciate that the present invention is not limited by the order of the additions in adding an anti-solvent. For example, a solution may be added to an anti-solvent or vice versa, though an embodiment may prefer one over the other. Crystallization of a compound is often better when a solution is added to the anti-solvent, but operationally it is often more convenient to add the anti-solvent to the solution. When adding an anti-solvent to a residue, the order of addition is of minimal relevance. The term combining encompasses both orders of addition.

Many processes of the present invention involve crystallization out of a particular solvent, i.e., obtaining a solid from a solution. Crystallization may occur spontaneosuly or be induced. The present invention covers both embodiments where crystallization or precipitation occurs spontaneously, or is induced/accelerated, unless if such inducement is critical for obtaining a particular polymorph.

The starting material used for the processes of the present invention may be any crystalline or other form of desloratadine, including various solvates and hydrates. With crystallization processes, the crystalline form of the starting material does not usually affect the final result since the original crystalline form is lost once a material goes into solution. With a slurry/trituration process, the starting material sometimes makes a difference, since without complete dissolution, the original crystal form may not be completely lost.

The desloratadine used may be obtained from loratadine, by hydrolysis of the carbamate, preferably under basic conditions. Loratadine itself may be prepared from N-methyl desloratadine by removing N-methyl group of N-methyl desloratadine by formation of the carbamate through reaction with a haloformate. The haloformate used may be an alkyl or aryl formate, with optional halogen substituted at first and/or second position of the formate, i.e., 2-chloroethyl-chloroformate. The carabmate may be prepared in an anhydrous C₅ to C₁₂ hydrocarbon, such as toluene. When N-methyl desloratadine is used as a stating material, loratadine may or may not be isolated in preparation of desloratadine.

The removal of the carbamate group of loratadine may be carried out with a base at elevated temperature. A preferred temperature is reflux temperature. A preferred base is an alkali metal or alkaline earth metal base such as potassium or sodium hydroxide. A preferred solvent is a C₁ to a C₄ alcohol such as 2-propanol.

The desloratadine from the reaction may then be recovered as a polymorphic form. In a preferred embodiment, the reaction mixture is distributed between an organic phase and water, resulting in desloratadine moving to the organic phase.

In one embodiment, desloratadine so prepared is crystallized out from a solution of ethyl acetate by seeding with a mixture of Form I and Form II to obtain a solid, followed by addition of a C₅ to C₁₂ aromatic or saturated hydrocarbons such as heptane. This embodiment also encompasses use of hydrocarbon as an anti-solvent, where crystallization happens after addition of the hydrocarbon (Seeding may be carried out before, during or after adding the hydrocarbon). See e.g. Example 25. A solid may then be recovered by techniques known in the art such as filtration and dried, preferably at about vacuum (Pressure below about 50 mmHg) and about room temperature. This embodiment produces from about a 4:1 to about a 1:3 mixture of Form I to Form II.

In another embodiment, desloratadine is crystallized out of a mixture of toluene and a C, to C₄ alcohol such as 2-propanol. See Example 28 and 29. In a preferred embodiment, a solution of desloratadine in heptane is concentrated and combined with 2-propanol, preferably the 2-propanol being less than 20% by volume. Crystallization may be carried out by heating the solution, preferably at a temperature above about 60° C. followed by addition of 2-propanol and cooling, preferably to a temperature below about 30° C. The crystals may be recovered by conventional techniques. The addition of relatively minor amounts of 2-propanol to toluene manipulates the ratio of Form I and II, and allows for a more facile crystallization. For example, when crystallization is carried out from toluene, substantially Form II is obtained, but addition of up to about 20% volume of a C₁ to C₄ alcohol as co-solvent allows for obtaining a mixture of the forms. This technique allows for carrying out much of the synthetic process in toluene, and yet obtain a mixture from toluene.

Pharmaceutical formulations of the present invention contain desloratadine Form I and/or Form II, optionally in mixture with other form(s) of desloratadine. The desloratadine prepared by the processes of the present invention are ideal for pharmaceutical composition. In addition to the active ingredient(s), the pharmaceutical compositions of the present invention may contain one or more excipients. Excipients are added to the composition for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellosel), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, desloratadine and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Capsules, tablets and lozenges, and other unit dosage forms preferably contain from about 2 to about 20 mg of desloratadine, more preferably about 2 mg to about 10 mg of desloratadine, and most preferably about 5 mg.

The mixtures of Form I and Form II for pharmaceutical formulations may be prepared by using a solution of desloratadine in a C₅ to C₁₂ aromatic hydrocarbons such as toluene. The concentration of desloratadine is preferably at least about 15% by weight. The solution is then combined with an anti-solvent, preferably a C₁ to C₄ alcohol such as isopropanol or methanol, more preferably in a ratio of about 7 to about 14% compared to the volume (v/v) of toluene. The resulting precipitate is then recovered by conventional techniques. Seeding to manipulate crystallization is optional.

In more detail, a solution of desloratadine in toluene is prepared. The concentration of desloratadine is preferably at least about 15% by weight. A salt of desloratadine may be used as starting material, particularly since salt formation may be used to purify the starting material. Suitable salts include the acetate.

When starting from a salt, depending on the solubility of the salt, the salt may be suspended in toluene as to form a slurry. A base is then added to the slurry to obtain the free acid, which is readily soluble in toluene, and moves into solution. Suitable bases include those of alkali metal and alkaline earth metals such as potassium, sodium and calcium oxide/hydroxide/carbonate, preferably sodium or potassium hydroxide.

The base is preferably added as an aqueous solution to the toluene, where two phases form. An about a 2% to about 6% solution of sodium or potassium hydroxide, preferably about a 4% solution may be used. The slurry is preferably heated to increase the reaction rate, to for example a temperature of about 40 to about 70° C. The resulting two phase reaction system is preferably stirred at this temperature until complete dissolution.

The reaction results in neutralization of the salt, leading to solution of desloratadine free acid in toluene. After phase separation, such as by physical means with use of a separatory funnel, the toluene solution of desloratadine may be washed with distilled water at the same temperature to obtain more of the acid before discarding the aqueous phase.

In one embodiment, the resulting toluene solution is concentrated by vacuum or at atmospheric pressure jacket: preferably about 55° C. to about 130° C.) to dryness, though it is theoretically possible to precipitate the acid by reducing the solubility of the solvent.

The solid material is then dissolved in a mixture of toluene- and a C₁ to C₄ alcohol, such as isopropanol, methanol and 2-propanol, preferably 2-propanol, in the ratio of about 5:1 to about 15:1, more preferably about 9:1. The addition of relatively minor amounts of 2-propanol (anti-solvent) to toluene manipulates the ratio of Form I and II, and allows for a more facile crystallization. The mixture is preferably warmed to increase its solubility, such as to a temperature of about 50 to about 70° C., more preferably about 60° C. The warm solution is then preferably cooled to a temperature of about 10° C. to about 30° C., more preferably to about 20° C. The cooling may be carried out slowly, during a span of few hours. Cooling in about 4 hours is optimal. The cooled solution is then preferably stirred for a few hours, more preferably of about 5 to about 8 hours.

After cooling, partial precipitation occurs. This slurry solution is preferably warmed again, to about 45 to 55° C., and dropped into cold n-heptane, preferably at about −5 to about +5° C. The precipitated solid material is then recovered preferably by filtration, and dried. Drying may be carried out at ambient or reduced pressure. In one embodiment, drying is carried our in a vacuum oven at about 25-35° C. overnight.

Instrumentation

X-Ray powder diffraction data were obtained using by method known in the art using a SCINTAG powder X-Ray diffractometer model X'TRA equipped with a solid state detector. Copper radiation of 1.5418 Å was used. A round aluminum sample holder with round zero background quartz plate, with cavity of 25(diameter)*0.5(dept) mm.

DSC analysis was done using a Mettler 821 Stare. The weight of the samples was about 5 mg; the samples were scanned at a rate of 10° C./min from 30° C. to 250° C. The oven was constantly purged with nitrogen gas at a flow rate of 40 ml/min. Standard 40 μl aluminum crucibles covered by lids with 3 holes were used.

IR analysis was done using a Perkin Elmer SPECTRUM ONE FT-IR spectrometer in DRIFTt mode. The samples in the 4000-400 cm⁻¹ interval were scanned 64 times with 4.0 cm⁻¹ resolution

In the following examples, the vacuum oven used had a pressure of approximately 30 mm Hg and the refrigerator had a temperature of about 5° C.

Preparation of desloratadine Form I

### EXAMPLE 1

Preparation of Desloratadine Form I— Dry

A solution of desloratadine (5.0 grams) in acetonitrile (54 ml) was heated at reflux temperature until complete dissolution. Charcoal (0.52 grams) was added to the clear solution, heated for a few minutes, and filtered. After filtration, the solution was heated again to reflux temperature, and allowed to cool to room temperature, resulting in crystallization. The resulting precipitate was filtered, and washed with 10 ml of ethyl acetate-n-hexane (3:1). The wet solid product was dried in a vacuum oven at room temperature. The X-Ray Powder diffraction showed that the sample had crystallized in polymorphic Form 1 (4.01 g). [The solvent to solute ratio is preferably from about 8 to about 14 ml/g]

### EXAMPLE 2

Preparation of Desloratadine Form I— Dry

A solution of desloratadine (5.0 grams) in dimethylformamide (19 ml) was heated at 75° C. until complete dissolution. Charcoal (0.55 grams) was added to the solution, heated for a few minutes, and filtered. After filtration, the solution was heated again, and allowed to cool to room temperature, allowing for crystallization. The resulting precipitate was filtered, and washed with 10 ml of ethyl acetate: n-hexane (1:1). The wet solid product was then dried in a vacuum oven at 40° C. The X-Ray Powder Diffraction pattern showed that the sample was crystallized in polymorphic Form I (3.33 g). [The solvent to solute ratio is preferably from about 3 to about 10 ml/g]

### EXAMPLE 3

Preparation of Desloratadine Form I—Wet and Dry

A solution of desloratadine (3.0 grams) in tetrahydrofuran (7.5 ml) was heated at reflux temperature (66° C.) until complete dissolution. The clear solution was allowed to cool to room temperature, resulting in crystallization. The resulting precipitate was filtered and was analyzed by X-Ray Powder Diffraction, and found to be Form I. The wet solid product was dried in a vacuum oven at room temperature. The X-Ray Powder diffraction showed that the sample had crystallized in polymorphic Form 1 (1.73 g). [The solvent to solute ratio is preferably from about 1 to about 5 ml/g]

### EXAMPLE 4

Preparation of Desloratadine Form I—Wet and Dry

A solution of desloratadine (3.0 grams) in diethyl carbonate (15 ml) was heated at reflux temperature (126° C.) until complete dissolution. The clear solution was allowed to cool to room temperature, resulting in crystallization. The resulting precipitate was filtered and was analyzed by X-Ray Powder Diffraction, and found to be Form I. The wet solid product was dried in a vacuum oven at room temperature. The X-Ray Powder diffraction showed that the sample had crystallized in polymorphic Form 1 (2.32 g). [The solvent to solute ratio is preferably from about 2 to about 8 ml/g].

Preparation of a Mixture of Desloratadine Form I and Form II in which Form II was about 2-10%

### EXAMPLE 5

Preparation of Desloratadine Form I—Wet and Dry

Desloratadine (3.0 grams) in chloroform (15 ml) was dissolved at room temperature, resulting in a clear solution. n-hexane was added to the solution until complete precipitation. The resulting mixture was kept in the refrigerator overnight. The resulting precipitate was filtered and was analyzed by X-Ray Powder Diffraction, and found to be Form I. A trace of Form II was present in the sample, as evident by XRPD pattern. The wet solid product was then dried in a vacuum oven at room temperature for about 15 hours. The X-Ray Powder Diffraction Pattern showed that the sample had crystallized in polymorphic Form 1 (1.85 g). A trace of Form II was present in the sample as evident by XRPD (approximately 2% wt/wt Compared to Form I). [The solvent (chloroform) to solute ratio is preferably from about 2 to about 8 ml/g].

### EXAMPLE 6

Preparation of desloratadine Form I—Wet and Dry

Desloratadine (3.0 grams) in chloroform (15 ml) was dissolved at room temperature to obtain a clear solution. Di-isopropyl ether was added to the solution until complete precipitation. The resulting mixture was kept in the refrigerator. The resulting precipitate was filtered and analyzed by X-Ray Powder Diffraction and found to be Form I. A trace amount of Form II was present in the sample as evident by the XRPD pattern. The wet solid product was then dried in a vacuum oven at room temperature for about 15 hours. The X-Ray Powder Diffraction pattern showed that the sample had crystallized in polymorphic Form I. (1.35 g). A trace amount of Form II was also present in the sample as evident by XRPD. (approximately 6% wt/wt Compared to Form I). [The solvent (chloroform) to solute ratio is preferably from about 2 to about 8 ml/g].

### EXAMPLE 7

Preparation of Desloratadine Form I—Wet and Dry

A solution of desloratadine (3.0 grams) in absolute ethanol (13 ml) was stirred at room temperature until complete dissolution. Water was added to the clear solution until complete precipitation, and the resulting mixture was kept in the refrigerator overnight. The resulting precipitate was filtered and analyzed by X-Ray Powder Diffraction and found to be Form I. A trace of Form II was present in the sample as evident by XRPD pattern. The wet solid product was then dried in a vacuum oven at room temperature for about 15 hours. The X-Ray Powder Diffraction pattern showed that the sample was crystallized in polymorphic Form I. (1.64 g). A trace of Form II was present in the sample as evident by XRPD. (approximately 4% wt/wt Compared to Form I). [The solvent (ethanol) to solute ratio is preferably from about 2 to about 6 ml/g].

Preparation of a Mixture of Desloratadine Form I and Form II in the Ratio of Approximately 3-6 to 1, i.e., about 15-25% of Form II in the Form I.

### EXAMPLE 8

Preparation of a Mixture of Desloratadine Form I and II

A solution of desloratadine (5.0 grams) in ethyl acetate (25 ml) was heated at reflux temperature until complete dissolution. The warm solution was added to 50 ml of cold n-hexane (−13° C.) resulting in precipitation. The mixture was kept at −13° C. for 1 hour and filtered. The wet solid product was dried in a vacuum oven at room temperature overnight. The X-Ray Powder Diffraction Pattern showed that the sample had crystallized as a mixture of polymorphic Form I and Form II in the ratio of approximately 4 to 1. [The solvent (ethyl acetate) to solute ratio is preferably from about 3 to about 10. The ratio of ethyl acetate to hexane is preferably from about 1 to about 5 ml/g].

### EXAMPLE 9

Preparation of a Mixture of Desloratadine Form I and II

A solution of desloratadine (3.0 grams) in isobutyl acetate (15 ml) was heated at reflux temperature (120° C.) until complete dissolution. The clear solution was allowed to cool to room temperature, resulting in crystallization. The resulting precipitate was filtered and was analyzed by X-Ray Powder Diffraction, and found to be mixture of Form 1 and Form 2. The wet solid product was dried in a vacuum oven at room temperature. The X-Ray Powder diffraction showed that the sample had crystallized in polymorphic Form 1 and Form 2 in the ratio of approximately 5.5 to 1 (2.13 g). [The solvent to solute ratio is preferably from about 3 to about 8 ml/g].

Preparation of a Mixture of Desloratadine Form I and Form II in the Ratio of Approximately 1 to 1

### EXAMPLE 10

Solution of desloratadine (3.0 grams) in isopropanol (6 ml) was heated at reflux temperature (80° C.) until complete dissolution. The solution was allowed to cool to room temperature, resulting in crystallization of a solid material. The solid material was filtered and analyzed by X-Ray powder diffraction and found to be a mixture of Form 1 and Form 2 (Form 2 was 5-6% in the Form 1). The wet solid product was dried in a vacuum oven at room temperature overnight. The X-Ray Powder diffraction pattern showed that the sample was crystallized in mixture of polymorphic Form 1 and Form 2 in the ratio of approximately 1 to 1 (1.5 grams).

### EXAMPLE 11

Solution of desloratadine (10.0 grams) in isopropanol (25 ml) was heated at reflux temperature (80° C.) in a glass reactor until complete dissolution. The warm solution was cooled to 0° C. during 30 minutes resulting in crystallization of a solid material. The solid material was filtered and analyzed by X-Ray powder diffraction and found to be a mixture of Form 1 and Form 2 (Form 2 was 2% in the Form 1). The wet solid product was dried in a vacuum oven at room temperature overnight. The X-Ray Powder diffraction pattern showed that the sample was crystallized in mixture of polymorphic Form 1 and Form 2 in the ratio of approximately 1 to 1 (8.7 grams).

### EXAMPLE 12

Solution of desloratadine (3.0 grams) in 2-methyl-1 propanol (isobutanol) (3 ml) was heated at reflux temperature (110° C.) until complete dissolution. The warm solution was allowed to cool to room temperature resulting in crystallization of a solid material. The solid material was filtered and analyzed by X-Ray powder diffraction and found to be a mixture of Form 1 and Form 2 (Form 2 was 3% in the Form 1). The wet solid product was dried in a vacuum oven at room temperature overnight. The X-Ray Powder diffraction pattern showed that the sample was crystallized in mixture of polymorphic Form 1 and Form 2 in the ratio of 1 to 1 (2.7 grams).

### EXAMPLE 13

Preparation of a Mixture of Desloratadine Form I and II

Desloratadine (3.0 grams) in chloroform (5 ml) was heated at 50° C. until complete dissolution. n-hexane was added to the warm clear solution until complete precipitation. The resulting mixture was allowed to cool to room temperature, and kept in the refrigerator overnight and filtered. The filtered material was analyzed by X-Ray Powder Diffraction, which showed a mixture of Form I and Form II. (Form II was 38% in the Form 1). The wet solid product was dried in a vacuum oven at room temperature for about 15 hours. The X-Ray Powder Diffraction pattern showed that the sample had crystallized as a mixture of polymorphic Form I and Form II in the ratio of 1.5 to 1 (2.32 g). [The solvent to solute ratio is preferably from about 1 to about 5 ml/g].

### EXAMPLE 14

Preparation of a Mixture of Desloratadine Form I and II

Desloratadine Form 1 (1.5 grams) was ground in a mortar. After 1 hour, a sample was taken, and the X-Ray Powder diffraction pattern of the sample showed that the sample had crystallized as a mixture of polymorphic Form II and Form I (Form II was approximately 65 wt/wt Compared to Form I). Another sample taken after 1.5 hours showed, according to X-Ray Powder Diffraction, that the sample had crystallized as a mixture of polymorphic Form II and Form I. (Form II was approximately 63 wt/wt Compared to Form I).

Preparation of Desloratadine Form II

### EXAMPLE 15

Preparation of Form II

Desloratadine (5.0 grams), in the solid state, was heated at a temperature of 160° C. until all of the solid material had melted. The resulting clear liquid was allowed to cool slowly to room temperature, which solidified. The resulting solid material was ground in a mortar. The X-Ray Powder Diffraction pattern showed that the sample had crystallized in polymorphic Form II.

### EXAMPLE 16 (wet and dried)

Preparation of Desloratadine Form II—Wet and Dry

A solution of desloratadine (3.0 grams) in dimethyl carbonate (26 ml) was heated at reflux temperature (90° C.) until complete dissolution. The clear solution was allowed to cool to room temperature, resulting in crystallization. The resulting precipitate was filtered and was analyzed by X-Ray Powder Diffraction, and found to be Form II. The wet solid product was dried in a vacuum oven at room temperature. The X-Ray Powder diffraction showed that the sample had crystallized in polymorphic Form 11 (2.14 g).

### EXAMPLE 17

Preparation of Desloratadine Form I and Form II

A solution of desloratadine (20 grams) in iso-butyl acetate (120 ml) was heated in the glass reactor at 100° C. until complete dissolution. In another glass reactor the mixture of form I and form II of desloratadine (2 g) was suspended in n-heptane (80 ml) at 35° C. The hot iso-butyl acetate solution was dropped into n-heptane slurry (temperature of slurry was between 30-40° C.). The resulting crystalline product was filtered and was dried in a vacuum oven at room temperature. The X-Ray Powder Diffraction showed that the sample had crystallized in as a mixture of polymorphic Form I and Form II (16 g). Mixture of Form I and Form II was in the ratio of 37 to 63. [The solvent (isobutyl acetate) to solute ratio is preferably from about 6 to about 1 ml/g. The ratio of iso-butyl acetate to n-heptane is preferably from about 3:2 (v/v)]

### EXAMPLE 18

Preparation of Desloratadine Form I and Form II

A solution of desloratadine (400 grams) in iso-butyl acetate (2400 ml) was heated in the glass reactor at 100° C. until complete dissolution. In another glass reactor the mixture of form I and form II of desloratadine (40 g) was suspended in n-heptane (1600 ml) at 35° C. The hot iso-butyl acetate solution was dropped into n-heptane slurry (temperature of slurry was between 30-40° C.). The resulting crystalline product was filtered and was dried in a vacuum oven at room temperature for about 15 hours. The X-Ray Powder diffraction showed that the sample had crystallized in as a mixture of polymorphic Form I and Form 11 (353 g). Form I and Form II was in the ratio of 42 to 58. [The solvent (iso-butyl acetate) to solute ratio is preferably from about 6 to about 1 ml/g. The ratio of iso-butyl acetate to n-heptane is preferably from about 3:2 v/v]

### EXAMPLE 19

Preparation of Desloratadine Form I and Form II

A solution of desloratadine (10.0 grams) in isobutyl acetate (60 ml) was heated at 100° C. until complete dissolution. The hot isobutyl acetate solution was dropped into n-heptane at 35° C. (temperature of n-heptane was between 30-40° C.). The resulting crystalline product was filtered and was dried in a vacuum oven at room temperature. The X-Ray Powder Diffraction pattern showed that the sample was crystallized in as a mixture of polymorphic Form I and Form II (6.0 g) in the ratio of 63 to 37. [The solvent (isobutyl acetate) to solute ratio is preferably from about 6 to about 1 ml/g. The ratio of isobutyl acetate to n-heptane is preferably from about 3:2]

### EXAMPLE 20

Preparation of Desloratadine Form I and Form II

A solution of desloratadine (10.0 grams) in iso-butyl acetate (60 ml) was heated at 100° C. until complete dissolution. In another glass reactor the mixture of form I and form II of desloratadine (10 g) was suspended in iso-butyl acetate (60 ml) at 40° C. The hot iso-butyl acetate solution was dropped into iso-butyl acetate slurry (temperature of slurry was between 30-50° C.). The resulting crystalline material was filtered and was dried in a vacuum oven at room temperature. The X-Ray Powder diffraction showed that the sample had crystallized in as a mixture of polymorphic Form I and Form II (13.7 g). Form I and Form II was in the ratio of 59 to 41. [The solvent to solute ratio is preferably from about 1 to about 6 ml/g].

### EXAMPLE 21

Preparation of Desloratadine Form I and Form II

A solution of desloratadine (10.0 grams) in 10% iso-propanol-iso-butyl acetate (50 ml) was heated at 100° C. until complete dissolution. The clear solution was allowed to cool to room temperature, resulting in crystallization. The resulting precipitate was filtered and was dried in a vacuum oven at room temperature. The X-Ray Powder diffraction showed that the sample had crystallized in as a mixture of polymorphic Form I and Form II. (6.1 g) in the ratio of 56 to 44. [The solvent to solute ratio is preferably from about 5 to about 1 ml/g].

### EXAMPLE 22

Preparation of Desloratadine Form I and Form II

Desloratadine (20.0 grams) in 10%-iso-propanol—toluene (50 ml) was dissolved at 80° C. until complete dissolution. The clean solution was allowed to cool to room temperature, resulting in crystallization. The crystalline material was filtered and was dried in a vacuum oven at room temperature for about 15 hours. The X-Ray Powder Diffraction Pattern showed that the sample had crystallized in as a mixture of polymorphic Form I and Form 11 (8.9 g) in the ratio of 50 to 50. [The solvent (10% iso-propanol-toluene) to solute ratio is preferably from about 2.5 to about 1 ml/g].

### EXAMPLE 23

Preparation of Desloratadine Form I and Form II

Desloratadine (10.0 grams) in 5% methanol-toluene (20 ml) was dissolved at 100° C. to obtain a clear solution. The resulting solution was allowed to cool to 20° C. for 30 minutes. The resulting crystalline material was filtered and dried in a vacuum oven at room temperature for about 16 hours. The X-Ray Powder Diffraction pattern showed that the sample had crystallized in as a mixture of polymorphic Form I and Form II (5.7 g) in the ratio of 53 to 47. [The solvent (methanol-toluene) to solute ratio is preferably from about 2 to about 1 ml/g.]

### EXAMPLE 24

Preparation of Desloratadine Form I and Form II

Desloratadine (32.5 grams) in mixture of iso-propanol (6 ml) and ethyl acetate (200 ml) was dissolved at 80° C. to obtain a clear solution. Charcoal (1.0 gram) was added to the clear solution, heated for a few minutes, and filtered. After filtration, the crystallization started immediately from solution. The resulting crystalline material was filtered and was dried in a vacuum oven at room temperature for about 16 hours. The X-Ray Powder Diffraction pattern showed that the sample had crystallized in as a mixture of polymorphic Form I and Form 11 (23.3 g) in the ratio of 74 to 26.

### EXAMPLE 25

Preparation of Desloratadine as a Mixture of Polymorphs from N-Methyl Desloratadine [with Loratadine as an Intermediate]

N-methyl desloratadine (40 g) was dissolved in 200 ml anhydrous toluene at 60° C. A mixture of 30 ml ethyl chloroformate and 60 ml anhydrous toluene was added dropwise for 60 minutes. After the addition had been completed, the reaction was checked for conversion. Because of the incomplete conversion, a mixture of 2.5 ml ethyl chloroformate and 5 ml anhydrous toluene was added dropwise for 10 minutes. The reaction mixture was filtered off and the filtrate was concentrated at reduced pressure. Repeated co-distillation with 2-propanol resulted in the removal of toluene. The residue was dissolved in 150 ml 2-propanol and heated to reflux. In the meantime, 16 g KOH was added. When the reaction mixture reached the reflux temperature, another 16 g KOH was added. After 1 h another 8 g KOH was added and the mixture was heated for 1 h. The reaction was checked for conversion, and since the conversion was incomplete, 8 g KOH was added to the mixture and it was refluxed for an additional hour. The reaction mixture was cooled to 60° C. and 60 ml water was added. After the solid had been dissolved, the two phases of the liquid were separated. The lower (aqueous) phase was extracted twice with 100 ml ethylacetate. These ethylacetate extracts were combined and used to dissolve the residue that was obtained by the concentration of the original upper (2-propanol) phase to about half of its original weight. This was washed successively with 150 ml water, 100 ml water and 100 ml brine prior to drying over 20 g K₂CO₃. The drying agent was filtered off and the filtrate was seeded with a mixture of Form I and II, and combined slowly with 350 ml n-heptane with stirring. Crystallization started slowly, after 1-2 hours the suspension was taken to refrigerator. The resulting crystalline product was filtered off and dried in a vacuum oven at room temperature. Yield: 14.20 g (40%). The X-ray Powder Diffraction measurement showed that the ratio of the two polymorphic forms was 62:38 (Form 1/Form 2).

### EXAMPLE 26

Preparation of Desloratadine as a Mixture of Polymorphs from Loratadine

Loratadine (40 g) and KOH (16 g) were suspended in 150 ml 2-propanol and heated to reflux. Half an hour later than the reaction mixture reached the reflux temperature, another 16 g KOH was added. After 1 hour, another 8 g KOH was added and the mixture was heated for 1 hour. The reaction was checked for conversion. The conversion was complete. The reaction mixture was cooled to 60° C., and 60 ml water was added. After the solid had dissolved, the two phases of the liquid were separated. The lower (aqueous) phase was extracted twice with 100 ml ethylacetate. These ethylacetate extracts were combined and used to dissolve the residue that was obtained by the concentration of the original upper (2-propanol) phase to about half of its original weight. This was washed successively with 150 ml water, 100 ml water and 100 ml brine. The combined aqueous washings were extracted with 50 ml ethylacetate which was added to the other ethylacetate extract. This was dried over 20 g K₂CO₃, decanted and dried again over 20 g K₂CO₃. The drying agent was filtered off and the filtrate was seeded and after 15 minutes it was combined slowly with 150 ml n-heptane with stirring overnight at room temperature. The resulting crystalline product was filtered off and dried in a vacuum oven at room temperature. Yield: 12.85 g (40%). The X-ray Powder Diffraction measurement showed that the ratio of the two polymorphic forms was 77:23 (Form 1/Form 2).

### EXAMPLE 27

Preparation of Desloratadine as a Mixture of Polymorphs from Loratadine

Loratadine (40 g) and KOH (16 g) was suspended in 150 ml 2-propanol and heated to reflux. Half an hour after reaching reflux temperature, another 16 g KOH was added. After 1 h, another 8 g KOH was added and the mixture was heated for another 1 hour. The reaction was checked for conversion. The conversion was complete. The reaction mixture was cooled to 60° C. and 60 ml water was added. After the solid had been dissolved, the two phases of the liquid were separated. The lower (aqueous) phase was extracted twice with 100 ml ethylacetate. These ethylacetate extracts were combined and used to dissolve the residue that was obtained by the concentration of the original upper (2-propanol) phase to about half of its original weight. This was washed successively with 150 ml water, 100 ml water and 100 ml brine. The combined aqueous washings were extracted with 50 ml ethylacetate which was added to the other ethylacetate extract. This was dried over 20 g K₂CO₃, decanted and dried again over 20 g K₂CO₃. The drying agent was filtered off and the filtrate was concentrated at 50° C. under vacuum to obtain a relatively concentrated solution (estimated concentration: 25 m/m %). This was seeded with a mixture of Form I and Form II, and after 15 minutes it was combined slowly with 60 ml n-heptane with stirring overnight at room temperature. The resulting crystalline product was filtered off and dried in a vacuum oven at room temperature. Yield: 21.13 g (65%). The X-ray Powder Diffraction measurement showed that the ratio of the two polymorphic forms is 43:57 (Form 1/Form 2).

### EXAMPLE 28

Preparation of Desloratadine as a Mixture of Polymorphs from Loratadine

To 2-propanolic (750 ml) solution of loratadine (200 g), KOH (80 g) was added while stirring, and heated to reflux (jacket: 95° C.) temperature. After 60 minutes, KOH (80 g), and after 120 minutes, KOH (40 g), were added to the reaction mixture. When the reaction was complete, the mixture was cooled to 50° C. and diluted with distilled water (300 ml). The two-phase mixture was stirred until complete dissolution of the solids. The lower (aqueous) phase was removed and the 2-propanolic phase was evaporated at vacuum (jacket: 50° C.). The resulting material was diluted with toluene (800 ml) and washed successively with distilled water (2×300 ml) at 50° C. From the resulting solution, 200 ml of solvent was evaporated at vacuum (jacket: 50° C.) to 150 ml residual volume. The residual volume was heated to 80° C., and 2-propanol (4.5 ml) was added, and cooled to 20° C. for 1 hour. After the combining and cooling, the crystalline material was filtered off and dried in vacuum at 25° C. The X-Ray Powder Diffraction showed that the sample had crystallized as a mixture of polymorphic Form I and Form II (15.6 g).

Mixture of Form I and Form II was in the ratio of 39 to 61.

### EXAMPLE 29

Preparation of Desloratadine as a Mixture of Polymorphs from Loratadine

To 2-propanolic (750 ml) solution of Loratadine (200 g) KOH (80 g) was added during stirring and heated to reflux (jacket: 95° C.). After 60 minutes KOH (80 g), 120 minutes KOH (40 g) were added to the reaction mixture. When the reaction was complete (after 5 hours), mixture was cooled to 50° C. and diluted with distilled water (300 ml). Two-phase mixture was stirred until complete dissolution of the solids. The lower (aqueous) phase was removed and the 2-propanolic phase was evaporated at vacuum (jacket: 50° C.). The resulting material was diluted with toluene (800 ml) and washed successively with distilled water (2×300 ml) at 50° C. From the resulting solution 150 ml solution was evaporated at vacuum (jacket: 50° C.) to 95 ml residual volume, which was then heated to 90° C. 2-propanol (10 ml) was added and cooled to 20° C. and kept at this temperature for 30 minutes. A crystalline material was filtered off and dried in vacuum at 25° C. The X-Ray Powder Diffraction showed that the sample had crystallized in as a mixture of polymorphic Form I and Form II (9.8 g). Mixture of Form I and Form II was in the ratio of 25 to 75.

### EXAMPLE 30

Preparation of Desloratadine Mixture Form I and Form II

A slurry of desloratadine acetate (20 grams) in toluene (100 ml) and 3.8% KOH solution (95,6 ml) was heated and stirred in the glass reactor at 60° C. until complete dissolution.

After phase separation, toluene solution of desloratadine was washed with distilled water (60 ml) at 60° C. The resulting toluene solution was concentrated by vacuum jacket: 60° C.) to dryness. The solid material was dissolved in toluene-2-propanol 9:1 (74 ml) at 60° C. The warm solution was cooled to 20° C. for 4 hours and stirred at this temperature for 8 hours. This slurry was warmed again to 45° C. In another glass reactor n-heptane (100 ml) was cooled to 0° C. The warm slurry of desloratadine in toluene was dropped into cold n-heptane (temperature of slurry was between 0-12° C.), and it was stirred at 0° C. for 1 hours. The resulting crystalline product was filtered and dried in a vacuum oven at room temperature. The X-Ray Powder Diffraction showed that the sample had crystallized in as a mixture of polymorphic Form I and Form II (ratio 76-24). (13.2 g, 79%, HPLC purity: 99.9%).

### EXAMPLE 31

Preparation of Desloratadine Mixture Form I and Form II

Desloratadine was prepared from desloratadine acetate according to the example 1. The X-Ray Powder Diffraction showed that the sample had crystallized in as a mixture of polymorphic Form I and Form II (10.8 g, 65%, HPLC purity: 99.8%). Mixture of Form I and Form II was in the ratio of 42 to 38.

### EXAMPLE 32

Preparation of Desloratadine Mixture Form I and Form II

A slurry of desloratadine acetate (20 grams) in toluene (100 ml) and 3.8% KOH solution (75 ml) was heated and stirred in the glass reactor at 60° C. until complete dissolution. After phase separation, toluene solution of desloratadine was washed with distilled water (60 ml) at 60° C. The resulting toluene solution was concentrated by vacuum (jacket: 60° C.) to dryness. The solid material was dissolved in toluene-2-propanol 9:1 (50 ml) at 60° C. The warm solution was cooled to 20° C. for 4 hours and stirred at this temperature for 10 hours. This slurry was warmed again to 50° C. The resulting toluene slurry was concentrated by vacuum (jacket: 50° C.) to half of volume. In another glass reactor n-heptane (100 ml) was cooled to 0° C. The warm slurry of desloratadine in toluene was dropped into cold n-heptane (temperature of slurry was between 0-12° C.), and it was stirred at 0° C. for 1 hours. The resulting crystalline product was filtered and was dried in a vacuum oven at room temperature. The X-Ray Powder Diffraction showed that the sample had crystallized in as a mixture of polymorphic Form I and Form II (64 to 36%) (10.6 g, 63%) HPLC purity: 99.7%.

Having thus described the invention with reference to particular preferred embodiments and illustrative examples, those in the art would appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used as a guidance. All references mentioned herein are incorporated in their entirety.

What is claimed is:

1. A process for preparing crystalline desloratadine Form I comprising the steps of:
a) preparing a solution of desloratadine in a solvent selected from the group consisting of acetonitrile, di-methyl formamide, tetrahydrofuran and diethylcarbonate, wherein desloratadine Form I crystallizes out of the solution; and
b) recovering the desloratadine Form I.

2. The process of claim 1, wherein the solvent is acetonitrile.

3. The process of claim 1, wherein the solvent is di-methyl formamide.

4. The process of claim 1, wherein the solvent is tetrahydrofuran.

5. The process of claim 1, wherein the solvent is diethylcarbonate.

6. The process of claim 1, further comprising a drying step.

7. The process of claim 1, wherein the solution is cooled to about 20° C. to about 30° C.

8. The process of claim 1, wherein the recovered Form I is substantially free of Form II.

9. The process of claim 8, wherein the ratio of Form II to Form I is less than about 1% by weight.

10. A process for preparing crystalline desloratadine Form I comprising the steps of:
a) preparing a solution of desloratadine in a solvent selected from the group consisting of chloroform and ethyl acetate;
b) combining the solution with an anti-solvent to precipitate desloratadine Form I; and
c) recovering desloratadine Form I.

11. The process of claim 10, wherein the anti-solvent is a C₂ to a C₈ ether.

12. The process of claim 11, wherein the ether is di-isopropyl ether.

13. The process of claim 12, wherein the solvent is chloroform.

14. The process of claim 13, wherein the ratio of desloratadine Form II to Form I is about 6%.

15. The process of claim 10, wherein the anti-solvent is a C₅ to a C₁₂ saturated hydrocarbon.

16. The process of claim 15, wherein the hydrocarbon is hexane.

17. The process of claim 16, wherein the solvent is chloroform.

18. The process of claim 17, wherein the solution has an initial temperature of at least about 40° C.

19. The process of claim 18, wherein the ratio of desloratadine Form II to Form I is about 35 to about 40% wt/wt.

20. The process of claim 17, wherein the solution has an initial temperature of less than about 40° C.

21. The process of claim 20, wherein the ratio of desloratadine Form II to Form I is about 2% wt/wt.

22. The process of claim 16, wherein the solvent is ethyl acetate.

23. The process of claim 22, wherein the ratio of desloratadine Form II to Form I is about 2% wt/wt.

24. A process for preparing crystalline desloratadine Form I comprising the step of:
a) preparing a solution of desloratadine in a C₁ to C₄ alcohol;
b) combining the solution with water to precipitate desloratadine Form I; and
c) recovering desloratadine Form I.

25. The process of claim 24, whereint the alcohol is ethanol.

26. The process of claim 24, wherein the Form I obtained has from about 2% to about 10% Form II.

27. A process for preparing crystalline desloratadine Form I comprising the steps of:
a) preparing a solution of desloratadine in isopropanol, wherein desloratadine Form I precipitates from the solution; and
b) recovering the desloratadine Form I.

28. The process of claim 27, wherein further comprising the step of seeding with Form II to increase ratio of Form II to Form I.

29. A process for preparing crystalline desloratadine Form II comprising the steps of:
a) melting desloratadine to obtain a molten material;
b) cooling the molten material to obtain a solid; and
c) grinding the solid.

30. A process for preparing a mixture of crystalline desloratadine Form I and Form II comprising the step of grinding crystalline desloratadine Form I.

31. A process for preparing crystalline desloratadine Form II comprising the steps of:
a) preparing a solution of desloratadine in dimethyl carbonate, wherein desloratadine Form II precipitates from the solution; and
b) recovering the desloratadine.

32. The process of claim 31, wherein the desloratadine Form II recovered is substantially free of Form I.

33. A process for preparing crystalline desloratadine Form I comprising the steps of:
a) preparing a solution of desloratadine in i-butyl acetate, wherein Form I precipitates from the solution; and
b) recovering the precipitate.

34. The process of claim 33, wherein the precipitate contains from about 15% to about 25% Form II.

35. A process for preparing crystalline desloratadine Form I comprising the steps of:
a) preparing a solution of desloratadine in a solvent selected from the group consisting of isopropanol and i-butanol, wherein desloratadine Form I precipitates from the solution; and
b) recovering the mixture.

36. The process of claim 35, wherein the solvent is isopropanol.

37. The process of claim 36, wherein the mixture contains less than about 10% Form II compared to Form I by weight.

38. A process for preparing a mixture of crystalline Form I and Form II of desloratadine comprising the step of drying desloratadine Form I crystals obtained by crystallization from a C₁ to a C₄ alcohol.

39. The process of claim 38, wherein the alcohol is isopropanol.

40. The process of claim 38, wherein the alcohol is isobutanol.

41. A process for making a mixture of crystalline desloratadine Form I and Form II comprising the steps of combining a solution of desloratadine in a suitable solvent with an anti-solvent containing seeds of both Form I and Form II of desloratadine to precipitate the mixture, and recovering the mixture.

42. The process of claim 41, wherein the mixture contains from about 35% to about 65% Form I by weight.

43. The process of claim 41, wherein the solvent is iso-butyl acetate.

44. The process of claim 41, wherein the antisolvent is a C₅ to C₁₂ hydrocarbon.

45. The process of claim 44, wherein the hydrocarbon is heptane.

46. A process for preparing a mixture of desloratadine crystalline Forms I and II containing at least about 25% of both of the Forms comprising the steps of:
a) preparing a solution of desloratadine in a solvent selected from the group consisting of ethyl acetate and iso-butyl acetate, in a mixture with about 3% to about 20% C₁ to C₄ alcohol by volume, wherein the mixture of Form I and II precipitates from the solution; and
b) recovering the mixture.

47. The process of claim 46, wherein the mixture contains at least about 40% of both forms by weight.

48. The process of claim 46, wherein the alcohol is present in about 10% by volume.

49. The process of claim 46, wherein the alcohol is selected from the group consisting of methanol, iso-propyl alcohol and mixtures thereof.

50. A process for preparing a mixture of crystalline desloratadine Form I and II comprising the steps of:
a) preparing a solution of desloratadine in iso-butyl acetate;
b) combining the solution with a C₅ to C₁₂ aromatic hydrocarbon to precipitate the mixture, wherein the combining may be carried out before, after or during crystallization; and
c) recovering the mixture.

51. The process of claim 50, wherein the hydrocarbon is heptane.

52. The process of claim 50, wherein the mixture contains from about 60% to about 70% Form I by weight.

53. The process of claim 50, further comprising increasing ratio of Form II to Form I by seeding the solution with a mixture of Form I and Form II before crystallization.

54. The process of claim 53, wherein the seeding results in about 35% to about 45% Form I compared to Form II by weight.

55. A process for preparing a mixture of crystalline desloratadine Form I and II comprising the steps of:
a) preparing a solution of desloratadine in iso-butyl acetate;
b) combining the solution with iso-butyl acetate at a temperature lower than the solution to crystallize the mixture; and
c) recovering the mixture.

56. The process of claim 55, further comprising seeding the solution with a mixture of Form I and Form II before crystallization.

57. A process for preparing a mixture of crystalline desloratadine Form I and Form II comprising the steps of:
a) preparing a solution of desloratadine in ethyl acetate;
b) seeding the solution with a mixture of Form I and Form II;
c) combining the solution with a C₅ to C₁₂ saturated hydrocarbon, wherein the combining may be carried out before, after or during crystallization; and
d) recovering the mixture of desloratadine Form I and II.

58. The process of claim 57, wherein the hydrocarbon is heptane.

59. The process of claim 57, wherein the mixture is about a 4:1 to about a 1:3 mixture of Form I to Form II wt/wt.

60. A process for preparing a mixture of crystalline desloratadine Form I and Form II comprising the steps of:
a) preparing a solution of desloratadine in 2-propanol and toluene, wherein the mixture of Forms I and II precipitates from the solution; and
b) recovering the mixture.

61. The process of claim 60, wherein precipitation occurs as a result of cooling the solution.

62. The process of claim 60, wherein ratio of 2-propanol to toluene is less than about 20% by volume.

63. The process of claim 60, wherein precipitation occurs as a result of adding a C₅ to C₁₂ saturated hydrocarbon as an anti-solvent.

64. The process of claim 63, wherein the anti-solvent is n-heptane or n-hexane.

65. The process of claim 63, further comprising the step of seeding the solution.

66. A process for preparing a mixture of Form I and Form II, comprising the steps of:
a) providing a first solution of desloratadine in toluene;
b) evaporating the toluene to obtain a residue;
c) dissolving the residue in a mixture of toluene and a C₁ to C₄ alcohol to obtain a second solution;
d) cooling the second solution to obtain a slurry;
e) combining the slurry with a C₅ to C₁₂ saturated hydrocarbon to precipitate the mixture; and
f) recovering the mixture.

67. The process of claim 66, wherein the alcohol is 2-propanol.

68. A process for preparing a mixture of desloratadine Form I and Form II comprising the steps of:
a) combining desloratadine acetate, toluene and KOH to obtain a reaction mixture;
b) heating the mixture, whereby two phases are obtained;
c) separating the phases;
d) concentrating the separated organic phase;
e) dissolving the obtained concentrate in a toluene-2-propanol mixture containing less than about 20% 2-propanol by volume;
f) cooling the solution to obtain a slurry;
g) combining the slurry with cold n-heptane; and
h) recovering mixture of desloratadine forms I and II.

69. The stable mixture of claim 68, wherein the process further comprises washing the product of step c with water.

70. The stable mixture of claim 68, wherein the process further comprises warming the product of step f to 45° C.

71. The process of claim 68, wherein the mixture is about a 24 to about a 40% Form II compared to Form I.

72. A process for preparing crystalline desloratadine Form II comprising the steps of crystallizing desloratadine from toluene, and recovering the crystalline form.
